(19) United States
(12) Patent Application Publication
(10) Pub. No.: US 2021/0289821 A1
(43) Pub. Date: Sep. 23, 2021

(54) VITAMIN D2-RICH MUSHROOM POWDER, AND PREPARATION AND USES THEREOF

(71) Applicant: Hanpeng QU, .Changsha (CN)

(72) Inventors: Yun LIANG, Changsha (CN); Sheng CAO, Changsha (CN); Shenjian WANG, Changsha (CN)

(21) Appl. No.: 17/343,102

(22) Filed: Jun. 9, 2021

Related U.S. Application Data
(63) Continuation of application No. PCT/CN2019/101028, filed on Aug. 16, 2019.

(30) Foreign Application Priority Data
Dec. 10, 2018 (CN) 201811506520.4

(51) Int. Cl. A23L 5/30; A23L 31/00; A23L 33/155; A23L 3/42; A23P 10/22; A61K 31/593; A61K 36/06

(52) U.S. Cl. CPC A23L 5/36; A23L 31/00; A23L 33/155; A23L 3/42; A61K 2236/17; A61K 31/593; A61K 36/06; A23V 2002/00; A61K 2236/15; A23P 10/22

(57) ABSTRACT

A method for preparing a vitamin D₂-rich mushroom powder, including: slicing a mushroom to obtain mushroom slices, irradiating the mushroom slices with an ultraviolet light, drying the mushroom slices in nitrogen to obtain dried mushroom slices, and pulverizing the dried mushroom slices to obtain the vitamin D₂-rich mushroom powder. The ultraviolet light irradiation is performed at an ambient temperature of 20-55° C. and an ambient relative humidity of 50-85%, and a moisture content of the mushroom slices is not less than 20%.

### CROSS-REFERENCE TO RELATED APPLICATIONS

This application is a continuation of International Patent Application No. PCT/CN2019/101028, filed on Aug. 16, 2019, which claims the benefit of priority from Chinese Patent Application No. 201811506520.4, filed on Dec. 10, 2018. The content of the aforementioned applications, including any intervening amendments thereto, is incorporated herein by reference.

### TECHNICAL FIELD

This application relates to food processing, and more particularly to a vitamin D₂-rich mushroom powder, and a preparation and uses thereof.

### BACKGROUND

Vitamin D is a fat-soluble steroid derivative necessary for the human body, which can regulate the nutritional function of calcium and phosphorus metabolism and can prevent tumor, angiocardiopathy, autoimmune disease and diabetes. Vitamin D₂ and vitamin D₃ are closely related to health. Vitamin D₃ can be obtained by isomerizing 7-dehydrocholesterol in human epidermal cells after exposure to sunlight, while vitamin D₂ cannot be synthesized by the human body. The incidence of childhood rickets, rickets and senile osteoporosis is relatively high in China. Therefore, it is necessary to seek a variety of dietary approaches to increase the vitamin D₂ level in human body.

Vitamin D₂ is generally obtained from yeast fermentation method, penicillin waste mycelium extraction method and edible fungus conversion method, and has been applied to the pharmaceutical and food industries. The purity of vitamin D₂ used in the food industry is generally lower than that in the pharmaceutical industry. Therefore, the food industry often uses vitamin D₂ derived from edible fungi, and the flavor of edible fungi can be used at the same time.

In edible fungi, mushrooms are rich in vitamin D₂ and contain various nutrients. Therefore, edible fungi have been widely used as a source of vitamin D₂. However, the currently disclosed methods for producing mushroom powder containing vitamin D₂ requires drying the mushroom powder, and the vitamin D₂ will be converted into isomers or be degraded in the presence of oxygen in the high temperature drying process, resulting in low vitamin D₂ content and high by-product content in the obtained mushroom powder.

### SUMMARY

An object of this application is to provide a vitamin D₂-rich mushroom powder and a preparation method thereof to overcome the problems of low vitamin D₂ content and high by-product content in mushroom powder prepared in the prior art, which can increase the conversion rate of ergosterol into vitamin D₂ through controlling the wavelength of the ultraviolet light, temperature and humidity during the ultraviolet light irradiation process. This application adopts a drying method protected by nitrogen to avoid the formation of isomer impurities or degradation loss of vitamin D₂, improving the quality of the mushroom powder product.

To achieve the above object, the technical solutions of this application are described as follows.

In a first aspect, this application provides a method for preparing a vitamin D₂-rich mushroom powder, comprising:

(1) slicing a mushroom to obtain mushroom slices; irradiating the mushroom slices with an ultraviolet light; wherein the ultraviolet light is a combination of a 280-315 nm UVB and a 200-280 nm UVC; the ultraviolet light irradiation is performed at a temperature of 20-55° C. and a relative humidity of 50-85%; and a moisture content of the mushroom slices is not less than 20% during the ultraviolet light irradiation;

(2) drying the irradiated mushroom slices obtained in step (1) in nitrogen to obtain dried mushroom slices; and

(3) pulverizing the dried mushroom slices obtained in step (2) to obtain the vitamin D₂-rich mushroom powder.

In some embodiments, irradiation with the UVB is performed at an irradiation dose of 1.5-6.5 J/cm² for 8-150 min.

In some embodiments, irradiation with the UVC is performed at an irradiation dose of 80-120 mJ/cm² for 20-30 min.

In some embodiments, the ultraviolet light irradiation is performed on two sides of the mushroom slices.

In some embodiments, in step (1), the mushroom slices have a thickness of 0.8-1.2 mm.

In some embodiments, in step (2), the drying is performed at 60-80° C. in a warm air drying oven.

In some embodiments, in step (3), the dried mushroom slices are subjected to superfine pulverization to obtain the vitamin D₂-rich mushroom powder with a particle size of 100-200 mesh.

In a second aspect, this application provides a vitamin D₂-rich mushroom powder prepared by the above method, wherein in the vitamin D₂-rich mushroom powder, a content of vitamin D₂ is more than or equal to 350 μg/g; the total number of colonies is less than or equal to 800 cfu/g; and no pathogenic bacteria are detected.

In a third aspect, this application provides a food the vitamin D₂-rich mushroom powder, wherein the food is a health food or a functional food.

Through the above technical solutions, this application has the following beneficial effects.

(1) The ergosterol contained in the mushroom can be converted into vitamin D₂ by irradiating the mushroom with ultraviolet light combined with UVB and UVC. UVC ultraviolet light can also sterilize the mushroom to ensure that the microbial indicators in the obtained mushroom powder meet the requirements of food or pharmaceutical processing.

(2) The ambient temperature and relative humidity in the ultraviolet light irradiation process can be controlled to avoid excessive drying of the mushroom, so as to increase the conversion rate of ergosterol in the mushroom into vitamin D₂ and further increase the vitamin D₂ content in the mushroom powder.

(3) The drying process of mushroom slice uses nitrogen protection to isolate the air and reduce the temperature of the drying process, effectively avoiding the formation of isomer impurities or degradation loss of vitamin D₂, so as to reduce the production of by-products.

### DETAILED DESCRIPTION OF EMBODIMENTS

The endpoints and any values of the ranges disclosed herein are not limited to the precise range or value, and these ranges or values should be understood to include values close to these ranges or values. For numerical ranges, each range between the end values of each range, between the end values of each range and individual point values, and between individual point values can be combined with each other to give one or more new numerical ranges, and such numerical ranges should be construed as specifically disclosed herein.

In a first aspect, this application provides a method for preparing a vitamin D₂-rich mushroom powder, which has the following steps.

(1) A mushroom is sliced to obtain mushroom slices, and the mushroom slices are irradiated with an ultraviolet light. The ultraviolet light is a combination of a 280-315 nm UVB and a 200-280 nm UVC. The ultraviolet light irradiation is performed at a temperature of 20-55° C. and a relative humidity of 50-85%. A moisture content of the mushroom slices is not less than 20% during the ultraviolet light irradiation.

(2) The irradiated mushroom slices obtained in step (1) are dried in nitrogen to obtain dried mushroom slices.

(3) The dried mushroom slices obtained in step (2) are pulverized to obtain the vitamin D₂-rich mushroom powder.

In this application, the mushroom can be Agaricus bisporus, Lentinus edodes, other mushroom species or a combination thereof. The mushroom needs to be refrigerated to be 15° C. or less within 2 days after being harvested The ultraviolet light is provided by a UVB lamp tube and a UVC lamp tube. The power of the UVB lamp tube is 50-100 W, and the power of the UVC lamp tube is 15-40 W. The mushroom is sliced and placed on a metal mesh tray, and the ultraviolet lamp tubes are fixed on the shelf which is located on both sides of the tray and 70-80 cm away from the tray, to ensure that the distance between the tray and the ultraviolet light is 10-60 cm for double-sided irradiation. The mushroom is dried by a hot air circulating drying oven, and the circulating air is filled with nitrogen with purity above 99.9%. The compressed air is provided by an oil-free air compressor, and the nitrogen is generated by a pressure swing adsorption nitrogen generator. An air outlet of the nitrogen generator is connected to an inlet of the circulating air of the hot air circulating drying oven for nitrogen filling and drying.

In some embodiments, irradiation with the UVB is performed at an irradiation dose of 1.5-6.5 J/cm² for 8-150 min. The intensity of the UVB light can be 1.5 J/cm², 2 J/cm², 2.5 J/cm², 3 J/cm², 3.5 J/cm², 4 J/cm², 4.5 J/cm², 5 J/cm², 5.5 J/cm², 6 J/cm², 6.5 J/cm² or any value between the two values.

In some embodiments, irradiation with the UVC is performed at an irradiation dose of 80-120 mJ/cm² for 20-30 min.

In some embodiments, in step (1), the mushroom slices have a thickness of 0.8-1.2 mm. The mushroom needs to be manually or mechanically cleaned before slicing to remove the residual medium.

In some embodiments, in step (2), the drying is performed at 60-80° C. in a warm air drying oven.

In some embodiments, in step (3), the dried mushroom slices are subjected to superfine pulverization to obtain the vitamin D₂-rich mushroom powder with a particle size of 100-200 mesh.

In a second aspect, this application provides a vitamin D₂-rich mushroom powder prepared by the above method. In the vitamin D₂-rich mushroom powder, a content of vitamin D₂ is more than or equal to 350 μg/g. The total number of colonies is less than or equal to 800 cfu/g, and no pathogenic bacteria is detected.

In a third aspect, this application provides a food containing the vitamin D₂-rich mushroom powder, and the food is a healthy food or a functional food.

The application will be further described below in detail with reference to the accompanying examples. In the following examples, the content of vitamin D₂ is measured by the method of GB14755-2010 using L-7000 high performance liquid chromatography (Hitachi, Japan). The total number of colonies is measured by the method of GB4789.2-2016, the Escherichia coli are measured by the method of GB4789.3-2016, and the pathogenic bacteria are measured by the method of GB 29921-2013. Agaricus bisporus and Lentinus edodes are commercial products of Shandong Linyi Ruize Agricultural Technology Co., Ltd.

### Example 1

(1) Slicing

Freshly-harvested Agaricus bisporus was cleaned to remove the residual medium. 10 kg of the cleaned Agaricus bisporus were sliced into Agaricus bisporus slices with a thickness of 0.8 mm.

(2) Ultraviolet Light Irradiation

Two sides of the Agaricus bisporus slices were irradiated with UVB with a wavelength of 280 nm at an irradiation dose of 1.5 J/cm² for 180 min and then irradiated with UVC with a wavelength of 200 nm at an irradiation dose of 80 mJ/cm² for 30 min, where the ultraviolet light irradiation was performed at an ambient temperature of 20° C. and an ambient relative humidity of 50%, and a moisture content of the Agaricus bisporus slices was maintained at 20%.

(3) Drying

The irradiated Agaricus bisporus slices were dried at 60° C. in nitrogen in a warm air drying oven to obtain dried Agaricus bisporus slices.

(4) Pulverization

The dried Agaricus bisporus slices were superfinely pulverized to obtain Agaricus bisporus powder with a particle size of 100 mesh.

### Example 2

(1) Slicing

Freshly-harvested Agaricus bisporus was cleaned to remove the residual medium. 10 kg of the cleaned Agaricus bisporus were sliced into Agaricus bisporus slice with a thickness of 1.2 mm.

(2) Ultraviolet Light Irradiation

Two sides of the Agaricus bisporus slices were irradiated with UVB with a wavelength of 300 nm at an irradiation dose of 4 J/cm² for 100 min and then irradiated with UVC with a wavelength of 240 nm at an irradiation dose of 100 mJ/cm² for 25 min, where the ultraviolet light irradiation was performed at an ambient temperature of 35° C. and an ambient relative humidity of 70%, and a moisture content of the Agaricus bisporus slices was maintained at 30%.

(3) Drying

The irradiated Agaricus bisporus slices were dried at 70° C. in nitrogen in a warm air drying oven to obtain dried Agaricus bisporus slices.

(4) Pulverization

The dried Agaricus bisporus slices were superfinely pulverized to obtain Agaricus bisporus powder with a particle size of 200 mesh.

### Example 3

(1) Slicing

Freshly-harvested Lentinus edodes was cleaned to remove the residual medium. 10 kg of the cleaned Lentinus edodes were sliced into Lentinus edodes slices with a thickness of 1.0 mm.

(2) Ultraviolet Light Irradiation

Two sides of the Lentinus edodes slices were irradiated with UVB with a wavelength of 315 nm at an irradiation dose of 6.5 J/cm² for 8 min and then irradiated with UVC with a wavelength of 280 nm at an irradiation dose of 120 mJ/cm² for 20 min, where the ultraviolet light irradiation was performed at an ambient temperature of 55° C. and an ambient relative humidity of 85%, and a moisture content of the Lentinus edodes slices was maintained at 40%.

(3) Drying

The irradiated Lentinus edodes slices were dried at 80° C. in nitrogen in a warm air drying oven to obtain dried Lentinus edodes slices.

(4) Pulverization

The dried Lentinus edodes slices were superfinely pulverized to obtain a Lentinus edodes powder with a particle size of 150 mesh.

### Example 4

(1) Slicing

Freshly-harvested Agaricus bisporus was cleaned to remove the residual medium. 10 kg of the cleaned Agaricus bisporus were sliced into Agaricus bisporus slices with a thickness of 0.8 mm.

(2) Ultraviolet Light Irradiation

Two sides of the Agaricus bisporus slices were irradiated with UVB with a wavelength of 305 nm at an irradiation dose of 8 J/cm² for 8 min and then irradiated with UVC with a wavelength of 200 nm at an irradiation dose of 80 mJ/cm² for 30 min, where the ultraviolet light irradiation was performed at an ambient temperature of 20° C. and an ambient relative humidity of 50%, and a moisture content of the Agaricus bisporus slices was maintained at 20%.

(3) Drying

The irradiated Agaricus bisporus slices were dried at 60° C. in nitrogen in a warm air drying oven to obtain dried Agaricus bisporus slices.

(4) Pulverization

The dried Agaricus bisporus slices were superfinely pulverized to obtain Agaricus bisporus powder with a particle size of 100 mesh.

### Example 5

(1) Slicing

Freshly-harvested Agaricus bisporus and Lentinus edodes were cleaned to remove the residual medium. 5 kg of the cleaned Agaricus bisporus and 5 kg of the cleaned Lentinus edodes were sliced into Agaricus bisporus slices and Lentinus edodes slices with a thickness of 0.8 mm.

(2) Ultraviolet Light Irradiation

Two sides of the Agaricus bisporus slices and the Lentinus edodes slices were irradiated with UVB with a wavelength of 290 nm at an irradiation dose of 3 J/cm² for 180 min and then irradiated with UVC with a wavelength of 220 nm at an irradiation dose of 150 mJ/cm² for 20 min, where the ultraviolet light irradiation was performed at an ambient temperature of 45° C. and an ambient relative humidity of 65%, and a moisture content of the Agaricus bisporus slices and the Lentinus edodes slices were maintained to 30%.

(3) Drying

The irradiated Agaricus bisporus slices and the Lentinus edodes slices were dried at 50° C. in nitrogen in a warm air drying oven to obtain dried Agaricus bisporus slices and dried Lentinus edodes slices.

(4) Pulverization

The dried Agaricus bisporus slices and the dried Lentinus edodes slices were superfinely pulverized to obtain Agaricus bisporus and Lentinus edodes powder with a particle size of 180 mesh.

### Example 6

(1) Slicing

Freshly-harvested Agaricus bisporus and Lentinus edodes were cleaned to remove the residual medium. 5 kg of the cleaned Agaricus bisporus and 5 kg of the cleaned Lentinus edodes were sliced into Agaricus bisporus slices and Lentinus edodes slices with a thickness of 1.2 mm.

(2)

Two sides of the Agaricus bisporus slices and the Lentinus edodes slices were irradiated with UVB with a wavelength of 290 nm at an irradiation dose of 1 J/cm² for 150 min and then irradiated with UVC with a wavelength of 260 nm at an irradiation dose of 110 mJ/cm² for 20 min, where the ultraviolet light irradiation was performed at an ambient temperature od 25° C. and an ambient relative humidity of 60%, and a moisture content of the Agaricus bisporus slices and the Lentinus edodes slices were maintained at 20%.

(3) Drying

The irradiated Agaricus bisporus slices and the Lentinus edodes slices were dried at 75° C. in nitrogen in a warm air drying oven to obtain dried Agaricus bisporus slices and a dried Lentinus edodes slices.

(4) Pulverization

The dried Agaricus bisporus slices and the dried Lentinus edodes slices were superfinely pulverized to obtain Agaricus bisporus and Lentinus edodes powder with a particle size of 180 mesh.

### Comparative Example 1

(1) Slicing

Freshly-harvested Agaricus bisporus was cleaned to remove the residual medium. 10 kg of the cleaned Agaricus bisporus were sliced into Agaricus bisporus slices with a thickness of 0.8 mm.

(2) Ultraviolet Light Irradiation

Two sides of the Agaricus bisporus slice were irradiated with UVB with a wavelength of 280 nm at an irradiation dose of 1.5 J/cm² for 100 min and then irradiated with UVC with a wavelength of 200 nm at an irradiation dose of 80 mJ/cm² for 15 min under normal temperature and pressure.

(3) Drying

The irradiated Agaricus bisporus slices were dried at 60° C. in nitrogen in a warm air drying oven to obtain dried Agaricus bisporus slices.

(4) Pulverization

The dried Agaricus bisporus slices were superfinely pulverized to obtain Agaricus bisporus powder with a particle size of 100 mesh.

### Comparative Example 2

(1) Slicing

Freshly-harvested Agaricus bisporus was cleaned to remove the residual medium. 10 kg of the cleaned Agaricus bisporus were sliced into Agaricus bisporus slices with a thickness of 1.2 mm.

(2) Two sides of the Agaricus bisporus slices were irradiated with UVB with a wavelength of 300 nm at an irradiation dose of 4 J/cm² for 65 min and then irradiated with UVC with a wavelength of 240 nm at an irradiation dose of 100 mJ/cm² for 10 min, where the ultraviolet light irradiation was performed at an ambient temperature of 35° C. and an ambient relative humidity of 70%, and a moisture content of the Agaricus bisporus slices were maintained at 30%.

(3) Drying

The irradiated Agaricus bisporus slices were dried at 120° C. in nitrogen in a warm air drying oven to obtain dried Agaricus bisporus slices.

(4) Pulverization

The dried Agaricus bisporus slices were superfinely pulverized to obtain Agaricus bisporus powder with a particle size of 200 mesh.

### Comparative Example 3

(1) Freshly-harvested Agaricus bisporus was cleaned to remove the residual medium. 10 kg of the cleaned Agaricus bisporus were sliced into Agaricus bisporus slices with a thickness of 1.2 mm.

(2) Two sides of the Agaricus bisporus slices were irradiated with ultraviolet light with a wavelength of 360 nm for 90 min under normal temperature and pressure.

(3) The irradiated Agaricus bisporus slices were subjected to freeze drying under vacuum at a sublimation temperature of 90° C., and the moisture content of the freeze-dried Agaricus bisporus slices was 3.4%.

(4) The freeze-dried Agaricus bisporus slices were superfinely pulverized to obtain Agaricus bisporus powder with a particle size of 150 mesh.

The mushroom powders prepared in Examples 1-6 and Comparative Examples 1-3 were measured for the vitamin D₂ content, the total number of colonies, Escherichia coli and pathogenic bacteria, and the measurement results were shown in Table 1.

TABLE 1
Measurement results of mushroom powders in Examples 1-6

| | Content of | Total number | Escherichia | |
|---|---|---|---|---|
| | vitamin D₂ | of colonies | coli | Pathogenic |
| Number | (μg/g) | (cfu/g) | (MPN/g) | bacteria/25 g |
| Example 1 | 615.3 | 561 | <0.3 | — |
| Example 2 | 630.8 | 453 | <0.4 | — |
| Example 3 | 611.7 | 358 | <0.3 | — |
| Example 4 | 354.8 | 610 | <0.5 | — |
| Example 5 | 458.1 | 587 | <0.4 | — |
| Example 6 | 432.7 | 679 | <0.4 | — |
| Comparative | 178.9 | 1130 | <0.6 | — |
| Example 1 | | | | |
| Comparative | 168.7 | 1253 | <0.8 | — |
| Example 2 | | | | |
| Comparative | 89.6 | 1317 | <0.9 | — |
| Example 3 | | | | |

It can be seen from Table 1 that the content of vitamin D₂ in the mushroom powder obtained by the preparation method of this application was significantly increased, the microbial indicators (total number of colonies and Escherichia co/i) were significantly better than those of the Comparative Examples, and pathogenic bacteria were not detected, meeting the food safety requirements. It can be seen from the comparison between Example 1 and Comparative Example 1 that by adjusting the environmental temperature and relative humidity during the ultraviolet light irradiation, the content of vitamin D₂ in mushroom powder was increased by 2.43 times, and the total number of colonies was reduced by 569 cfu/g. By comparing Example 2 and Comparative Example 2, it can be seen that when nitrogen was used to isolate oxygen and reduce the drying temperature, the content of vitamin D₂ was also significantly improved with respect to the mushroom powder obtained by high-temperature drying in oxygen. The content of vitamin D₂ in the mushroom powder obtained by the preparation method of this application can reach 630.8 μg/g.

The above are only the preferred embodiments of the present disclosure, and are not intended to limit the scope of the present disclosure. Any changes, modifications and improvements made by those skilled in the art without departing from the spirit of the present disclosure shall fall within the scope of the present disclosure.

What is claimed is:

1. A method for preparing a vitamin D₂-rich mushroom powder, comprising:
(1) slicing a mushroom to obtain mushroom slices; irradiating the mushroom slices with an ultraviolet light; wherein the ultraviolet light is a combination of a 280-315 nm UVB and a 200-280 nm UVC; the ultraviolet light irradiation is performed at a temperature of 20-55° C. and a relative humidity of 50-85%; and a moisture content of the mushroom slices is not less than 20% during the ultraviolet light irradiation;
(2) drying the irradiated mushroom slices obtained in step (1) in nitrogen to obtain dried mushroom slices; and
(3) pulverizing the dried mushroom slices obtained in step (2) to obtain the vitamin D₂-rich mushroom powder.

2. The method of claim 1, wherein irradiation with the UVB is performed at an irradiation dose of 1.5-6.5 J/cm² for 8-150 min.

3. The method of claim 1, wherein radiation with the UVC is performed at an irradiation dose of 80-120 mJ/cm² for 20-30 min.

4. The method of claim 1, wherein the ultraviolet light irradiation is performed on two sidesides of the mushroom slices.

5. The method of claim 1, wherein in step (1), the mushroom slices have a thickness of 0.8-1.2 mm.

6. The method of claim 1, wherein in step (2), the drying is performed at 60-80° C. in a warm air drying oven.

7. The method of claim 1, wherein in step (3), the dried mushroom slices are subjected to superfine pulverization to obtain the vitamin D₂-rich mushroom powder with a particle size of 100-200 mesh.

8. A vitamin D₂-rich mushroom powder prepared by the method of claim 1, wherein in the vitamin D₂-rich mushroom powder, a content of vitamin D₂ is more than or equal to 350 μg/g; the total number of colonies is less than or equal to 800 cfu/g; and no pathogenic bacteria are detected.

9. A food comprising the vitamin D₂-rich mushroom powder of claim 8, wherein the food is a health food or a functional food.
